(19) United States
(12) Patent Application Publication
(10) Pub. No.: US 2026/0357178 A1
(43) Pub. Date: Nov. 12, 2026

(54) BEAD MANUFACTURING DEVICE AND PROCESS

(71) Applicant: MOSA MEAT B.V., Maastricht (NL)

(72) Inventors: Eric Wolfgang WIELAND, Maastricht (NL); Nick VAN ZOMEREN, Maastricht (NL); Helge ZIGGERT, Maastricht (NL)

(73) Assignee: MOSA MEAT B.V., Maastricht (NL)

(21) Appl. No.: 19/490,430

(22) PCT Filed: May 17, 2024
(86) PCT No.: PCT/EP2024/063670

§ 371 Date: Dec. 5, 2025

(30) Foreign Application Priority Data
Jun. 9, 2023 (NL) 2035052

(51) Int. Cl. C12M 1/12; A23L 13/00; C12M 3/06

(52) U.S. Cl. CPC C12M 25/16; A23L 13/00; C12M 25/02; C12M 27/16

(57) ABSTRACT

The invention relates to a device and process to manufacture beads from a fluid, more specifically hydrogel beads encapsulating cells appropriate for cell culture.

### TECHNICAL FIELD

The invention is in the field of bead manufacturing, preferably hydrogel beads, preferably hydrogel beads for cell culture, more preferably hydrogel beads laden with animal cells appropriate for manufacture of cultured meat, more preferably cross-linkable hydrogel beads.

### BACKGROUND OF THE INVENTION

Bead manufacturing, specifically hydrogel beads, has been accomplished before. The closest prior art to the invention was identified as the paper “Continuous Production of Uniform Calcium Alginate Beads by Sound Wave Induced Vibration”, Lee et al., J. Chemical Tech. Biotechnol. 1996, 67, 255-259.

In Lee's paper, a speaker is used to produce sound waves, carried by air to a rubber membrane, the rubber membrane applying a force to a chamber filled with a hydrogel precursor fluid, the fluid being then ejected as beads or droplets through a single orifice located parallel to the centre of the membrane, the beads falling on a cross-linking solution and becoming hydrogel beads.

This prior art has several issues that prevent it from being adapted to industrial production, namely the low throughput present when only a single orifice is used. Changing this prior art to increase its bead producing capabilities is not a trivial task, since several problems appear: first, if more than one orifice is present along a radial direction, and considering that the membrane is restrained by its circumference at a certain radial distance from its centre, then the centre surface of the membrane will deform further than the surface near the restraining circumference, causing the volume of liquid displaced to change radially. This means beads produced in a central orifice will be larger/heavier than beads produced at other radial distances. This prevents the production of multiple homogenous beads at the same time.

Another issue identified by the inventors is that transmission of force to the membrane by air displacement is not efficient and is prone to variations in performance if a airtightness of the air chamber is not perfect before operation or degrades over time.

Another issue with this design, is that the membrane is free to oscillate, allowing for undesired harmonics of the target frequency to develop.

The inventors set out to solve at least these issues, in the manner of this invention, which replaces the air gap with a physical plunger, restrains the membrane with a contact cone to prevent unwanted vibrations or development of vibrations in harmonic frequencies, and corrects the uneven deformation of the membrane to the edges to allow for multiple bead output orifices to be present that can deliver homogeneous volumes of beads between themselves.

### SUMMARY OF THE INVENTION

The inventors describe a new bead manufacturing device.

The preferred embodiments of the invention are as follows:

In a preferred embodiment the invention is a bead manufacturing device comprising an actuator 101, means to transmit the vibration of the actuator to a membrane contact cone 105 that pushes a membrane 106 in contact with the fluid in a feed chamber 108.

In a further preferred embodiment the membrane contact cone 106 presents with a truncated cone like shape, with the larger base oriented towards the bottom of the device.

In a further preferred embodiment the membrane contact cone is placed inside a main body 104 presenting with a cavity with the inverse shape from the membrane contact cone, preventing it from travelling upwards.

In a further preferred embodiment the membrane contact cone contacts the membrane, pushing it down with every cycle of the actuator, while maintaining it with a flat surface when the base of the membrane contact cone is substantially flat.

In a further preferred embodiment the main body 104 and a feed chamber part 107 are connected to each other in such a way as to form a void in-between them, this void being a feed chamber 108 filled with the fluid.

In a further preferred embodiment the membrane 106 is present between the main body 104 and the feed chamber part 107.

In a further preferred embodiment the bottom of the feed chamber part 107 has nozzles 109, these nozzles being either a hole or orifice through the feed chamber part or being separate parts, such as hollow cylinders.

In a further preferred embodiment the fluid is a hydrogel precursor, such as sodium alginate, containing cells appropriate for cell culture, such as bovine satellite cells or bovine fibro adipogenic progenitors used in cellular agriculture, and the beads dropping into a crosslinking CaCl2 solution after formation cross-linking the sodium alginate into cell encapsulating alginate hydrogel beads.

In a preferred embodiment the invention is a process for production of beads using the device, comprising the steps of:
a. Providing a fluid to a feed chamber 108;
b. Continuously flowing the fluid from a fluid inlet 110 through the feed chamber 108 and out the nozzles 109;
c. Using an actuator to impart a vibration to a membrane contact cone 106 that transmits this vibration to a membrane 106 that creates an oscillating pressure increase and decrease in the feed chamber 107;
d. Disturbing the stream of fluid exiting the nozzles as to create a controlled stream of homogeneous beads.

In a further preferred embodiment the invention is a process wherein the fluid is a hydrogel precursor such as sodium alginate, containing cells appropriate for cell culture, such as bovine satellite cells or bovine fibro adipogenic progenitors used in cellular agriculture, with the extra step e) of the beads dropping into a crosslinking CaCl2 solution after formation, cross-linking the sodium alginate into cell encapsulating alginate hydrogel beads.

### DESCRIPTION OF THE DRAWINGS

FIG. 1—Section view of an exemplary bead manufacturing device according to the invention

FIG. 2—Exploded 3D perspective view of the device of FIG. 1.

### REFERENCE SIGNS

101—Actuator
102—Actuator mount
103—Plunger
104—Main body
105—Membrane contact cone (MCC)
106—Membrane
107—Feed chamber part (FCP)
108—Feed chamber
109—Nozzles
110—Fluid inlet
111—Fluid inlet channel
201—Attachment means

### DETAILED DESCRIPTION OF THE INVENTION

The invention described herein aims primarily to solve the problem of producing large quantities of beads, especially hydrogel beads, in a controlled, replicable way.

The emerging field of cultivated meat is bringing about a paradigm change in terms of needs to scale up current laboratory setups to industrial scales. One technology that has been shown to be successful in culturing cells, in a laboratory setting, is the encapsulation of animal cells in hydrogel beads, or other appropriate materials. There is thus a need to improve the existing laboratory scale systems to create beads to allow for future large-scale manufacturing of cultured meat.

Cultivated meat is a subfield of biotechnology where the meat of an animal, especially the fat and muscle components, is a product of a cell culture process and not processed from an actual living animal, for example in a slaughterhouse/butcher. Nowadays, the cultivated meat field is becoming well known and already requires little explanation. These techniques are sometimes synonymously referred to as “cellular agriculture”, “lab grown meat” or other known terms.

The animal may be any of those mammals usually consumed by humans, such as bovines, ovines or porcines, but is preferably a bovine. Chicken, duck, turkey, quail and other birds are also possible options. Fish and other marine animals are also possible targets for cultured meat.

The cells described in this invention are, in a non-limiting fashion, preferably for human consumption, more preferably primary cells, more preferably bovine primary cells, more preferably Satellite Cells (SC's), also known as myosatellite cells. Other well-known cell types for production of cultivated meat are Fibro Adipogenic Progenitor (henceforth FAP's) or any type of stem cells, either pluri or multipotent, or Induced Pluripotent Stem Cells (iPSCs). Appropriate cell lines may also be used, for example immortalized or otherwise selected for the purpose of cultured meat applications, by natural selection or direct genetic manipulation. Both FAP's and SC's have been extensively studied and their definition is well known both in the general biotechnology area and the particular cultivated meat area. For the avoidance of doubt, a short explanation of these types of cells follows:

FAP cells are known to support the activation and differentiation of muscle satellite cells and also for having the ability to differentiate into adipocytes. This adipocyte formation mechanism is widely regarded as a way to produce edible fat for cultivated meat applications. FAP's can be found in muscle tissue.

SC's are multipotent cells found in mature muscle and serve as precursors to skeletal muscle cells. SC's may contribute further satellite cells to the muscle tissue or may differentiate into skeletal muscle cells. SC's are known to be involved in muscle reparation processes.

The cultivated meat field has made extensive use of SC's in trying to obtain an edible cultivated meat product, also using cultivated fat (FAP's or other fat cells) to provide taste to the muscle portion of the final food. Any other type of cells used in cultured meat or human-appropriate food can be part of the invention such as mesenchymal stem cells, adult stem cells, pluripotent stem cells, myocytes, adipocytes, chondrocytes, or fibroblasts, for example.

Hydrogel beads for cell cultures are usually made by mixing a hydrogel precursor solution, for example sodium alginate, with live cells, then in some way formed into droplets that are immersed in a crosslinking solution, for example a CaCl₂ solution when the hydrogel precursor is sodium alginate, and then immersed in a cell culture medium to allow for further growth.

The inventors created a multiple, preferably hydrogel, bead manufacturing device based on mechanical vibrations.

The device of this invention relies on an actuator that causes a plunger to vibrate and transmit that vibration to a membrane through a membrane contact cone (MCC), the membrane in turn inducing a disturbance on a fluid that is flowing out from a feed chamber through one or more nozzles or orifices, producing controlled droplets of the same fluid.

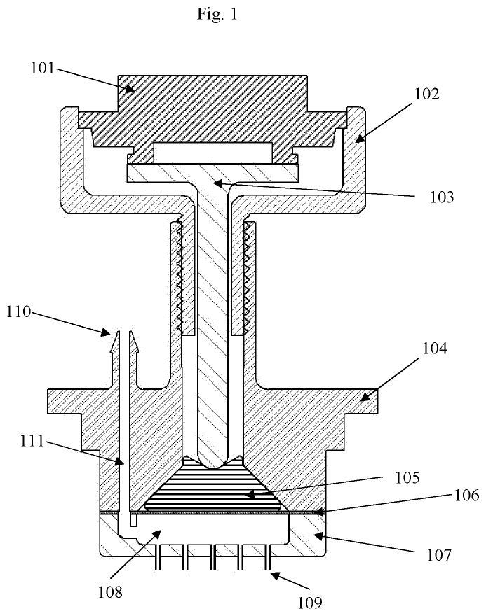

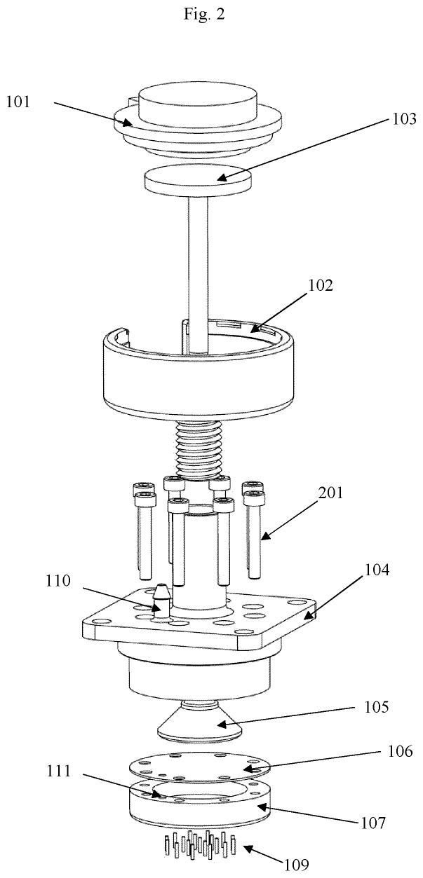

The workings of the invention will now be explained in more detail, with the help of FIGS. 1 and 2. In both FIG. 1 and FIG. 2, the top of the figure is considered to be the up direction, and the bottom the down direction. Top is also to mean higher, bottom to mean lower.

An actuator 101 is any device that causes a cycling movement in a part with a certain frequency and amplitude, or what can be seen as a mechanical vibration. Actuators may be for example of pressurized air type, where the increase and decrease of pressure creates a mechanical vibration that causes a part to move, or they can for example have a motor, such as any type of motor adapted to create reciprocating movement (a back-and-forth motion). A piezo electric actuator is also a known type of actuator that relies on the piezoelectric properties of a crystal, for example. Another type is a moving coil actuator, a type of linear motor with a fixed magnet and a coil that is free to move, used in this case to create a vertical movement by applying an alternating electrical current. The invention can work with any actuator that can react to the required amplitude and frequencies of movement of the membrane contact cone.

An actuator 101 (or a device with an equivalent function) is attached to an actuator mount 102 and in turn a plunger 103 is attached to the actuator in any of several possible ways, for example by the use of adhesive means, by using bolts and nuts, by screwing or any other common attachment means. The actuator mount may have several shapes, as long as it conforms to the function of holding the actuator in a fixed position relative to the device's main body 104, so that the coil or other moving parts may move freely and impart a required motion to the plunger.

The main body 104 and the actuator mount 102 are attached to each other in an immovable way (during operation), for example by any of several possible fastening means, for example by having flanged edges that are bolted together, or, as in the case of FIG. 1, by screwing the actuator mount into the main body.

The actuator mount 102 and the main body 104 both present with a linear cavity, along the height of the device, and these two cavities in combination accommodate the plunger 103 both at rest and when moving. The actuator mount may be provided with enough free space to allow any parts of the plunger that are not contained inside the linear cavity to move without contacting the actuator mount. What is meant is that the actuator mount has enough internal space to allow the plunger to move without developing impacts or friction and that the plunger is constrained between the actuator mount and the main body. In an alternative embodiment, the cavity may be tighter and low friction bushings or similar means may be provided to guide the plunger while minimizing friction. An example of an actuator mount with such necessary free space is shown in FIG. 1, where the widest horizontal section of the plunger is located at a certain vertical distance from the surface of the actuator mount.

The plunger translates the linear movement of the actuator to the membrane contact cone (MCC) 105. The MCC is shaped as a truncated cone with a larger base on the bottom and a smaller one on the top. The top base is arranged to accommodate the lower end of the plunger, for example by presenting with a flattened surface, an indentation, attachment means or a depression as the one seen in FIG. 1. The main body's bottom edge is shaped in such a way as to follow the outer profile of the MCC's cone shape, preventing it from travelling upwards. Basically, the main bottom presents with a cavity that is the inverse shape from the MCC. When at rest, the presence of the membrane 106, especially when under tension but also simply by its presence, prevents the MCC from travelling downwards, i.e., falling. In an alternative embodiment of the invention, the MCC and the plunger may be combined into a single part that provides all the equivalent necessary functions of the separate parts.

The membrane 106 is located between the main body 104 and the feed chamber part (FCP) 107. Preferably, the lowest part of the MCC and the bottom surface of the main body are flat in relation to each other. Thus, the membrane may stretch across the main body and the MCC without noticeable deformation, as long as the MCC is not pushing on it.

The FCP 107 presents with a cavity, located at its top surface, i.e., below the membrane when the FCP is attached to the main body. This cavity, when covered by the membrane 106, forms the feed chamber 108. The FCP and the main body may be attached by any of common attachment means, such as bolts and nuts or screws, such as depicted on FIG. 2, which shows attachment means, 201, fashioned as screws. For example, the membrane may be perforated at its edges to allow screws attaching the main body to the FCP to travel through it. This can be seen for example in FIG. 2, where the membrane presents with perforations. The feed cavity thus formed may be fed a fluid, such as an hydrogel precursor or any other material appropriate to manufacture a bead, optionally laden with cells appropriate for cell culture, by feeding through the fluid inlet channel 111, which attaches to an external source of fluid by connecting means applied to the fluid inlet 110, present on the main body. The main body and the FCP present with a hollow part that forms a continuous fluid inlet channel 110 extending from the fluid inlet 110 to the feed chamber 108.

The feed chamber part 107 presents with nozzles on its bottom surface. These nozzles may simply be through holes or orifices from the bottom surface up to the feed chamber 108 or comprise separate parts, such as hollow cylinders, of stainless steel or another appropriate material, forced or screwed into the same through holes. “Nozzle” is to be seen as meaning any holes, inserts or specially treated surfaces can allow the creation of a stream of beads or liquids.

In operation, an alternating current is supplied to the actuator 101 which causes the actuator to produce an oscillating up and down motion, moving the plunger 103 along with it. By means of the attachment means present to secure the actuator mount 102 to the main body 104, for example with the screw attachment shown in FIG. 1, the membrane may be pre-tensioned by the plunger as the actuator mount moves up or down in relation to the main body. Alternatively, the membrane may be pre-tensioned when mounting it between the main body and the feed chamber part 107 by stretching it as required. Alternatively, the membrane may also be pretensioned by having an actuator that is not returning to its theoretical zero position, but that is actually already pushing down on the plunger when not in cyclical operation. This may be achieved by mechanical means, or, in some actuators, by providing a base electric tension. The plunger pushes the membrane contact cone 105 downwards when the actuator forces it to do so, and the membrane, following the lower surface of the MCC, intrudes into the feed chamber 108 causing its volume to diminish, effectively further pushing out the flowing fluid present within it through the nozzles and inducing a disturbance in an otherwise stable flow. When the base of the contact cone presents with a flat bottom surface, the membrane does not deform in a bell shape, but instead proceeds downwards while keeping a flat shape. This allows the dislocation of fluid inside the feed chamber to be uniform across its horizontal section, which in turn means that the amount of fluid and pressure applied at each nozzle is the same. Without the MCC, for example by having a pressurized air cavity above the membrane, the membrane would deform in a bell shape due to the lateral constraints and dislocate more fluid in the central portion of the membrane/feed chamber, causing the pressure to increase in a non-uniform way across the interior fluid volume of the feed chamber. This would in turn cause the beads formed by multiple nozzles along a diameter of a, for example, circular shaped feed chamber, to differ in properties, especially volume. Optionally, when the membrane contact cone does not present with a flat bottom surface, for example presenting with a protrusion that does not press on the entire surface of the membrane, a bell shape is produced on the membrane. In this case, the dimensions of the feed chamber would need adjusting to allow the pressure distribution imbalance to become irrelevant at a point when the pressure waves have reached the bottom of the chamber, or the nozzles could be made in such a way that their diameter is small enough that a pressure restriction effect appears, effectively cancelling out the irregular pressure distribution created by the membrane. The stress applied to the membrane as the plunger pushes it down causes the membrane to in turn push the MCC up when the actuator is in a part of the cycle where it travels upwards, dragging the plunger with it. Without the membrane tension, the MCC would not travel upwards unless it was physically connected to the plunger, which is also a possibility, by gluing or any other appropriate attachment means. Alternatively, as described previously, the MCC and plunger may be a single part. The MCC cannot travel further upwards than allowed by the main body, which is shaped at its bottom as the inverse cone surface from the MCC, and as such prevents the membrane from oscillating fully or developing harmonics. The avoidance of upwards travel of the membrane also prevents ruptures by accidental overpressure of the fluid in the feed chamber when fed by the inlet channel, or during cleaning cycles, as described elsewhere in this specification. During this operation, the feed chamber is maintained full of flowing fluid and under appropriate pressure, through external means, such as reservoirs and pumps connected to the feed chamber by the fluid inlet 110 and through the fluid inlet channel 111. The flow of fluid through the system is controlled so that the vibration induced by the membrane (i.e., the small cycling pressure increase and decrease in the chamber) induces droplet formation some distance from the nozzles. Ideally, the fluid is in constant flow from the fluid inlet and out through the nozzles during operation and it's the mechanical disturbance induced by the membrane that causes homogeneous droplets to form. The theoretical underpinning of droplet formation from a stream of fluid are well known in the scientific literature, for example in Wheleahn, M.; et al, Microencapsulation using vibrating technology, Journal of Microencapsulation, 2011; 28(8): 669-688. The hydrodynamic system consisting of several nozzles with a certain diameter, the fluid feed pressure and flow into the chamber and the amplitude and frequency of the membrane pushing down during operation should be balanced in such a way that droplets only form when the membrane is in motion, and also that individual droplets do form and that an uncontrolled stream of fluid through the nozzles does not arise. The mathematics to simulate these events are readily available to the person knowledgeable in the field. Preferably, only one droplet forms per nozzle when the membrane pushes down into the feed chamber and back up (i.e., a cycle or a stroke). The volume of dislocated liquid is proportional to the vertical movement amplitude of the membrane, which is the same as the vertical dislocation of the plunger.

As a detail, it can be seen that the device is presented in the figures as having mostly circular symmetry. This means that, if the membrane was allowed to deform into a bell shape, then any nozzle along the same diameter of the base of the feed chamber part 107 could present with the same bead formation properties as any other nozzle.

A further advantage of the invention is that the membrane fully isolates the feed chamber from the electronic, electrical and main moving parts of the system, which allows for efficient cleaning and sterilization, a necessity in any biological application, especially food or feed. The system may be cleaned and sterilized by using Steam-in-Place (SIP) or Clean-in-Place (CIP) methods for example, by providing the steam or liquid through the fluid inlet channel, where it can proceed to the feed chamber and exit through the nozzles.

A further advantage of the invention is that the fluid in the feed chamber 108 is in direct mechanical contact with the actuator, with the force generated by the actuator being transmitted through the plunger, membrane contact cone and the membrane without the presence of an air gap or any other type of void volume that could cause substantial dampening or attenuation of the force provided by the actuator. This allows for a better control of the system's dynamic behaviour.

In the bead manufacturing device of the invention, the moving coil actuator or an equivalent function device should be connected to an external source of electrical current, in some cases oscillating. The voltage and optional oscillation frequency can vary greatly. These depend strongly on the properties of the membrane, the mass of all the moving parts, the pressure and volume of liquid in the feed chamber, the nozzle dimensions, the desired volume of the beads, etc. The system may be scaled up or down as desired (i.e. more or less nozzles, more or less volume of beads) as long as the operating parameters are adjusted. These adjustments can be made by an experienced operator by applying common engineering principles ex post facto of learning about this invention. An example of actual parameters is given in the exemplary embodiment of the invention below—these may be altered to conform to differing dimensions of the parts of the invention. In electrical terms, both DC or AC current may utilized, depending on the type of actuator used.

The fluid inlet 110 should be connected to an external source of fluid, such as a hydrogel precursor (for cross linkable hydrogels) or another fluid that is appropriate for cell culture, such as any type of extra cellular matrix like collagen. Preferably, this fluid is laden with cells, preferably cells for cell culture, which will then be encapsulated in the beads. In extremis, the device of the invention may be used to produce beads from any type of fluid and for any use.

The device may be mounted at the top of a vessel, such as a culture bioreactor and discharge directly into it or may be mounted atop any other type of appropriate vessel, including a transfer vessel for later transport to for example a culture bioreactor. The device may be transferred between several vessels or stay connected to a single one. For example, the device may be moved between bioreactors to fill each one with beads as required or be mounted to a vessel that serves as a transfer container to individual bioreactors, or any other foreseeable option. In case of a cross linkable hydrogel precursor, the hydrogel precursor beads exiting the nozzles should fall directly into an appropriate crosslinking solution. It may be the case that the device is mounted atop a bioreactor presenting with crosslinking solution that cross-links the hydrogel precursor beads and that crosslinking solution is later exchanged with culture medium. Preferably, the invention is used to produce cross-linkable hydrogel precursor beads, preferably laden with living cells, such as animal cells, appropriate for cell culture, preferably cells appropriate for the production of cultured meat, preferably muscle precursor cells or satellite cells, the cross-linkable hydrogel precursor being preferably sodium alginate, the cross-linking solution being preferably a CaCl₂ solution.

Beads with uncommon materials or properties are possible with this system, especially when working outside the cultured cells area, such as creating hot wax beads that could solidify by falling into a cooling bath, or frozen beads if made in a low enough temperature environment. Also, more uncommon cell types could be used, such as yeasts or even human cell for medical applications of the beads.

It can be easily seen that the device can produce high quantities of beads: for example, with a 200 Hz cycle and 10 nozzles, the device would make 120,000 beads per minute (60 s, times 200 Hz, times 10 nozzles). The scale up is linear: for example, upping the frequency to 100 Hz and the number of nozzles to 30, 1,800 thousand beads will be made per minute, or, 60 billion per hour. This definitely is a high throughput system, as needed for cultured meat manufacture, for example.

There are no hard material requirements for the device but some examples follow:

The actuator mount, the main body and the FCP may be made of steel, preferably stainless, or other metals or alloys. Other materials are possible, such as hard plastics, for example polyether ether ketone (PEEK), but preference is given to materials that can support the forces involved.

The plunger should in principle preferably be made from a stiff and light material, such as aluminium, its alloys, or a durable, rigid, plastic. Stainless steel can also be used. The plunger is a preferred part for controlling the mass of the system. It may be made thicker or slimmer, even hollow, to increase or decrease the mass as needed.

If the membrane contact cone and the plunger are built as a single part, then they will obviously be made from the same material.

The membrane should be made from a material that can oscillate, recover elastically, be put under tension for long periods, and maintain the fluid tightness of the feed chamber. Preference is given to silicone or rubber, or other polymers. Natural polymers may also be used, also any other materials currently used to produce membranes or elastic parts. A membrane made from a compliant or flexible metal could also be used.

The nozzles, when not just holes, may be made of metal, such as stainless steel or other alloys, or any other appropriate material, including plastics, ceramics or others.

### Exemplary Embodiment of the Invention

In an exemplary embodiment, used as a testing setup, the device of the invention is mounted atop a vessel containing a CaCl₂ solution. The actuator used is a moving coil actuator. The membrane contact cone presents with a flat lower surface, i.e., not inducing a bell shape on the deformed membrane at the maximum amplitude of movement in a cycle. A source of a sodium alginate laden with animal cells (the fluid) is connected to the fluid inlet and allowed to fill the feed chamber, at a flow rate of 12 ml/min per nozzle, with a total of 19 nozzles with an internal diameter of 500 microns.

The moving coil actuator is connected to a source of electrical current with a frequency of 450 hz and voltage of 11V (the voltage depends on the physical properties of a specific actuator). The actuator is selected to convert this current into a movement amplitude induced in the plunger/membrane system of approximately 18 microns a single bead per cycle/stroke (i.e. downwards movement of the plunger and membrane) with a spherical shape of 950 microns in diameter, which shrinks to 700 microns after crosslinking.

This effectively produces a number of hydrogel precursor beads per cycle equal to the number of nozzles, in this case 19, or 513,000 per minute. These beads, when falling into the cross-linking solution, become a hydrogel containing encapsulated animal cells. The cross-linking solution is then exchanged for culture media and cell culture proceeds in the same vessel, assuring a filter is present to retain the beads, or the beads are filtered out and transferred to a bioreactor. The bead manufacturing device of the invention may be retained atop the culture or removed from it.

It is to be understood that the current specification, besides describing a device for the manufacture of beads from a fluid, also discloses the implied or directly described associated process, such as duly claimed. Any technical features described are not limited to the literal disclosures but also imply obvious functional replacements.

1. A bead manufacturing device comprising an actuator 101, means to transmit the vibration of the actuator to a membrane contact cone 105 that pushes a membrane 106 in contact with a fluid in a feed chamber 108, wherein the membrane contact cone 106 presents with a truncated cone like shape, with the larger base oriented towards the bottom of the device and wherein the membrane contact cone is placed inside a main body 104 presenting with a cavity with the inverse shape from the membrane contact cone, preventing it from travelling further upwards than allowed by the main body.

2. The device according to claim 1 wherein the membrane contact cone contacts the membrane, pushing it down with every cycle of the actuator, while maintaining it with a flat surface when the base of the membrane contact cone is substantially flat.

3. The device according to claim 1, wherein the main body 104 and a feed chamber part 107 are connected to each other in such a way as to form a void in-between them, this void being a feed chamber 108 filled with the fluid.

4. The device according to claim 3, wherein the membrane 106 is present between the main body 104 and the feed chamber part 107.

5. The device according to claim 4, wherein the bottom of the feed chamber part 107 has nozzles 109, these nozzles being either a hole or orifice through the feed chamber part or being separate parts, such as hollow cylinders.

6. The device according to claim 1, wherein the fluid is a hydrogel precursor, such as sodium alginate, containing cells appropriate for cell culture, such as bovine satellite cells or bovine fibro adipogenic progenitors used in cellular agriculture.

7. (canceled)

8. (canceled)
